(19) United States
(12) Patent Application Publication
(10) Pub. No.: US 2004/0039252 A1
(43) Pub. Date: Feb. 26, 2004

(54) Self-navigating endotracheal tube

(76) Inventor: Kenneth Elmon Koch III, Waxhaw, NC (US)

Correspondence Address: KENNETH ELMON KOCH III, 409 BELVEDERE LANE, WAXHAW, NC, 28173, US

(21) Appl. No.: 10/462,059

(22) Filed: Jun. 16, 2003

Related U.S. Application Data
(60) Provisional application No. 60/391,889, filed on Jun. 27, 2002.

(51) Int. Cl. A61B 1/267

(52) U.S. Cl. 600/199

(57) ABSTRACT

The Self-Navigating Endotracheal Tube is an endotracheal tube that utilizes a light source and light measuring device to determine its location relative to a patient's trachea and esophagus. Using readings obtained via the light measurement device, a physician is capable of determining the proper insertion of the tube into a patient's trachea when a visual verification is not possible, as in the case of patients with spinal trauma, as well as the intubation of premature infants. It is also capable of providing an indication of self-extubation.

### CROSS-REFERENCE TO RELATED APPLICATIONS

This non-provisional patent application claims the benefit of U.S. Provisional Patent Application No. 60/391,889, filed Jun. 27, 2002.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The present invention relates to the medical field. More specifically, the present invention relates to an endotracheal tube that is capable of identifying its location relative to a patient's esophagus and/or trachea.

Currently, physicians utilize endotracheal tubes for the purpose of intubating patients. Intubation is used to assist with breathing during surgical procedures and/or trauma. Typically, in order to intubate a patient, the patient's head is titled back to allow the physician to see the patient's trachea. The tube is then inserted into the trachea of the patient and, subsequently, connected to a respirator.

In the event that spinal or neck trauma is suspected in the patient, a physician will refrain from tilting the patient's head backward to prevent the risk of additional injury. In this case, the physician must guess the location of the endotracheal tube once it is inserted into the patient's throat. This will henceforth be referred to as a blind intubation. In most of these cases the tube is inserted into the patient's esophagus. This presents the risk of pumping air into the patient's stomach, potentially resulting in vomiting. The self-navigating endotracheal tube is intended to provide the physician with an indication of the tube's location relative to the patient's esophagus and trachea. This indication will help ensure that the tube has been inserted properly into the trachea.

Another problem with current endotracheal tubes is the inability to verify their location without the use of an X-ray. Premature infants are often intubated and repeatedly X-rayed to verify the proper placement of the endotracheal tube. Many of these infants are exposed to multiple X-rays very early in their lives. The self-navigating endotracheal tube will eliminate the need to verify tube placement via an X-ray.

### BRIEF SUMMARY OF THE INVENTION

The Self-Navigating Endotracheal Tube is an endotracheal tube that is modified with a light source used in conjunction with a light measurement device. Both of these devices will be placed in, or on, the end of the endotracheal tube that is inserted into the patient's throat. They may either be fixed to the outside of the tube or inside the tube and capable of being removed once the tube has been placed in the trachea. The device works by measuring the resistance present in the photo-resistor when the light source is turned on and the tube is located in a patient's trachea or esophagus. The difference in resistance between the trachea and the esophagus allows a physician to determine the location of the tube.

### BRIEF DESCRIPTION OF THE SEVERAL VIEWS OF THE DRAWING

FIG. 1 is diagram of a Self-Navigating Endotracheal Tube illustrating its light source, light measurement device, and an opaque wall separating the two.

FIG. 2 is a close-up diagram illustrating the transmission of light between the light source and the light measurement device in the Self-Navigating Endotracheal Tube.

FIG. 3 is a close-up diagram illustrating the optimal configuration of the light source and the light measurement device on the end of the Self-Navigating Endotracheal Tube.

### DETAILED DESCRIPTION OF THE INVENTION

An endotracheal tube is a flexible tube that is used to intubate patients during surgery or when they are incapacitated and need assistance with breathing. It is inserted into a patient's trachea via the mouth. Some endotracheal tubes are also strung with a cord that runs the length of the tube and allows the physician to bend, or flex, the tube by pulling the cord. Proper location of an endotracheal tube is usually determined by tilting a patient's head back and visually verifying that the tube has been inserted into the trachea.

In the event that a patient has experienced spinal trauma, or is suspected of having experienced spinal trauma, verification is very difficult since the patient's head cannot be tilted for visual verification. This will be referred to as a blind intubation. What is needed is an endotracheal tube that is capable of identifying its location during a blind intubation.

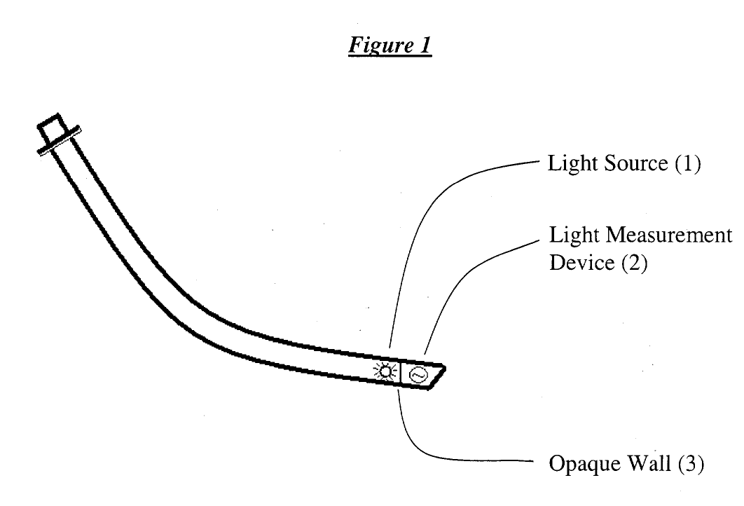

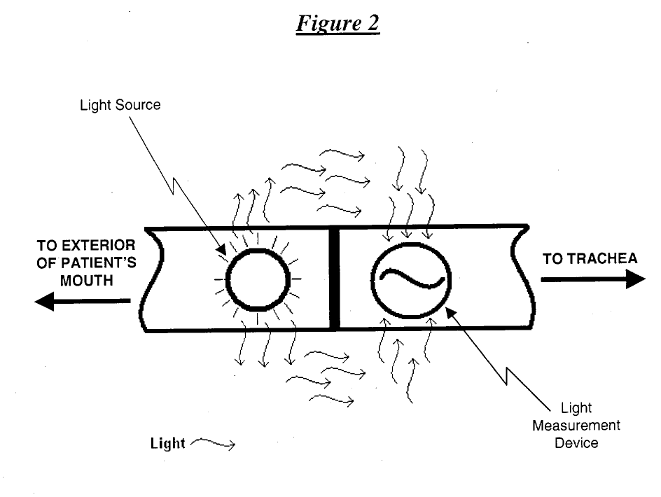

As shown in FIG. 1, the Self-Navigating Endotracheal Tube is an endotracheal tube that is modified with a light source 1 and a light measurement device 2 incorporated into the end of the tube that is inserted into a patient's trachea and/or esophagus. The tube is transparent, at least where the light source and the photo-resistor are housed, enabling light to pass through it. The light source 1 and photo-resistor 2 are separated by an opaque wall 3 that will not permit light to pass between the two unless it first passes through the wall of the tube to the outside and then back through the tube as shown in FIG. 2.

The Self-Navigating Endotracheal Tube relies on the fact that the esophagus is flaccid (collapsed) when empty and that the trachea is a rigid, hollow tube. When the Self-Navigating Endotracheal Tube is inserted into a patient's esophagus, the esophagus wraps closely around the exterior walls of the tube and thus prevents most of the light generated by the light source from exiting the tube. In contrast, the trachea forms an empty space around the tube and, subsequently, the tracheal cavity is illuminated, permitting light to exit the tube and reenter the tube at the photo-resistor. The difference in resistance between tracheal and esophageal placement of the Self-Navigating Endotracheal Tube allows a physician to determine the location of the tube.

The light source for the Self-Navigating Endotracheal Tube may consist of any type of device that is capable of delivering light in or around the end of the tube that is inserted into a patient including, but not limited to:

Incandescent, fluorescent, or halogen bulbs.

Laser generated light.

LEDs (light emitting diode).

Fiber optic cable delivering light from an external source including any of the sources listed above as well as natural light or sunlight.

The light source may be located inside the hollow interior of the tube, inside the walls of the tube, on the outside of the tube, or completely external to the tube as long as it is capable of delivering light to the end of the tube that is inserted into the patient.

The light measurement device may consist of any type of device or circuit that is capable of measuring the amount of light present around or in the end of the tube that is inserted into a patient including, but not limited to:

Photo-resistors

Solar cells

In addition, in the event that the light source and/or the light measurement device are located within the hollow interior of the tube, as shown in FIGS. 1 and 2, both the light source and the light measurement device may be capable of being removed once the tube has been placed in the trachea to ensure maximum airflow through the tube.

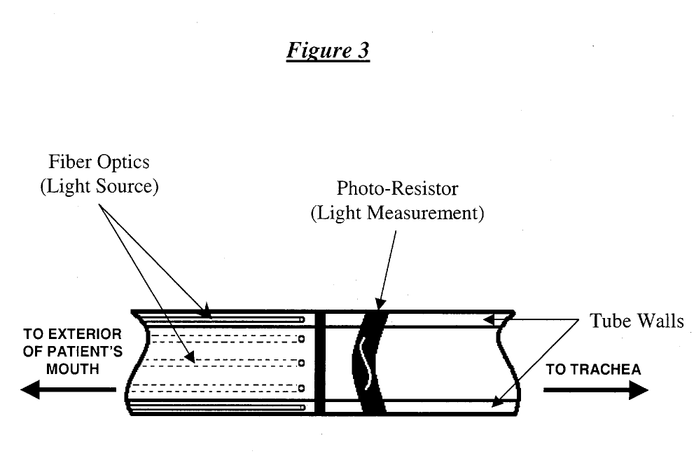

Ideally, the inventor envisions a tube with both the light source and light measurement device located on the outside wall of the tube, separated by and opaque section of tube as shown in FIG. 3. This would provide maximum usability in that it would not block airflow through the tube and allow the location of the tube to be verified at any time following initial placement. This configuration will also provide an indication of self-extubation (when patient movement causes the tube to exit the trachea), warning medical staff that tube needs to be re-positioned in the trachea.

In addition, the solution described above may be interfaced with a device, or plurality of devices, that translates the discrete esophageal and tracheal, or non-esophageal, measurements into a visible or audible indicator for a physician to see or hear, respectively.

What is claimed is:

1. An endotracheal tube that is capable of measuring the amount of light present at the end of the tube that is placed inside a patient, via any form of light measurement device located in or on the tube.

2. The endotracheal tube of claim 1, including a source of light that will illuminate the area immediately surrounding the end of the tube that is placed inside a patient and located in, on, or external to the tube.

3. The endotracheal tube of claim 2, where the end of the tube is transparent or translucent and allows light to pass through its walls, or sides.

4. The endotracheal tube of claim 3, containing a form of opaque barrier between the light measurement device of claim 1 and the light source of claim 2 that prevents light from being transmitted from the light source of claim 2 to the light measurement device of claim 1 unless the light passes outside of the tube and reenters via the transparent portion of the tube walls.

5. The endotracheal tube of claim 4, used with any device or circuit that will provide any form of audible, visual, or other indication of the status of the light measurement device of claim 1.
